(19) United States
(12) Patent Application Publication
(10) Pub. No.: US 2009/0266760 A1
(43) Pub. Date: Oct. 29, 2009

(54) FUNCTIONAL NUCLEIC ACIDS FOR BIOLOGICAL SEQUESTRATION

(76) Inventors: George William Jackson, Pearland, TX (US); Roger Joseph McNichols, Pearland, TX (US)

Correspondence Address: WINSTEAD PC, P.O. BOX 50784, DALLAS, TX, 75201, US

(21) Appl. No.: 12/044,737

(22) Filed: Mar. 7, 2008

Related U.S. Application Data
(60) Provisional application No. 60/905,792, filed on Mar. 8, 2007.

(51) Int. Cl. C02F 3/34; C12N 15/63; C12N 5/10

(52) U.S. Cl. 210/601; 435/320.1; 435/325; 435/455

(57) ABSTRACT

The present invention generally relates to methods of improving the removal and/or treatment of substances in bulk volumes, particularly to methods of improving the removal and/or treatment of contaminants in bulk volumes by nucleic acid interaction and by including such nucleic acid interactions in organisms. The present invention further relates to methods for generating and/or improving the interaction of nucleic acids with substances for removal and/or treatment. Bulk volumes may generally refer to any volume of substance wherein the removal and/or treatment of substances therein may occur. Nucleic acids may be utilized to bind and/or catalytically interact with substances in the bulk volume. Further, the nucleic acids may be included in an organism for sequestering substances within cells.

### CROSS-REFERENCE TO RELATED APPLICATIONS

This application claims the benefit of U.S. provisional patent application Ser. No. 60/905,792, filed Mar. 8, 2007, entitled “Aptamers, ribozymes, and other functional RNAs within non-coding RNAs for biological remediation or concentration”, the contents of which are hereby incorporated by reference

### BACKGROUND

The field of bioremediation has focused primarily on chemical transformation of contaminants as aided or catalyzed by microbes and/or plants (phytoremediation). However, the effects of complexation, adsorption, absorption, or any process otherwise resulting in sequestration or reduced mobility of contaminants may be just as important for many applications. Organisms have been widely selected and/or modified to aid in the treatment of waste products. Genetic engineering approaches have been applied to augment capabilities of these organisms. The selection and modifications have largely been focused on introducing and/or improving the catalytic capabilities of enzymes for breaking down and/or otherwise transforming wastes and contaminants. Enzymatic mechanisms thus remain the dominant means for breaking down or degrading substances using organisms.

### SUMMARY OF THE INVENTION

Various embodiments of the present invention are generally directed to novel selective nucleic acid ligands incorporated within non-coding nucleic acid sequences, capable of binding to or altering target molecules. Cells genetically manipulated to express such selective nucleic acid ligands are disclosed herein. The cells contemplated by the present invention include both prokaryotic as well as eukaryotic cells. The nucleic acid ligands encoded within a non-coding nucleic acid are either introduced into the cell using standard molecular biology techniques or are incorporated within the genomic non-coding nucleic acid of a cell by standard recombination techniques. Further contemplated is the use of such cells for sequestration of target molecules within the cells.

In various embodiments of this invention, the target molecules are contaminants present in bulk volumes. Bulk volumes include but are not limited to bodily wastes, municipal wastes, industrial effluents, bodies of fresh water, etc. In another embodiment, target molecules are valuable products that need to be reclaimed from bulk volumes. These valuable products include but are not limited to valuable metals, antibodies, drugs, hormones, proteins and pharmaceuticals.

Provided herein are embodiments of an expression vector comprising a chimeric gene encoding selective nucleic acid ligands within a non-coding nucleic acid, capable of binding to or altering target molecules, operatively linked to a functional promoter, where the vector when transfected in a host transcribes the chimeric gene.

Also, disclosed are embodiments of an isolated cell comprising the expression vector described supra.

Additionally, disclosed are embodiments of an isolated cell comprising at least one nucleic acid ligand sequence, incorporated into a genomic non-coding nucleic acid, where the nucleic acid ligand sequence binds to or catalytically alters a target molecule.

Provided herein are methods for sequestering within a cell a plurality of target molecules, present in a bulk volume comprising, generating a library of nucleic acid ligand sequences capable of binding to said target molecules; incorporating the nucleic acid ligand sequences in at least one non-coding nucleic acid within a cell; culturing the cell to achieve a cell population; contacting the cell population with the bulk volume; and separating the cell population from the bulk volume.

Furthermore, provided are methods for bioremediation of contaminants present in a bulk volume comprising, generating a library of nucleic acid ligand sequences capable of binding to or altering the contaminants; incorporating the nucleic acid ligand sequences in at least one non-coding nucleic acid in a cell; culturing the cell to achieve a cell population; contacting the cell population with the bulk volume; and separating the cell population from the bulk volume.

Other objects, features, and advantages of the present invention will be apparent to one of skill in the art from the following detailed description and figures.

### BRIEF DESCRIPTION OF THE FIGURES

In order that the manner in which the above recited and other enhancements and objects of the invention are obtained, a more particular description of the invention briefly described above will be rendered by reference to specific embodiments thereof, which are illustrated, in the appended drawings. Understanding that these drawings depict only typical embodiments of the invention and are therefore not to be considered limiting of its scope, the invention will be described with additional specificity and detail through the use of the accompanying drawings in which:

FIG. 1 illustrates an example of an insertion tolerant nucleic acid with an inserted sequence subjected to selective pressure.

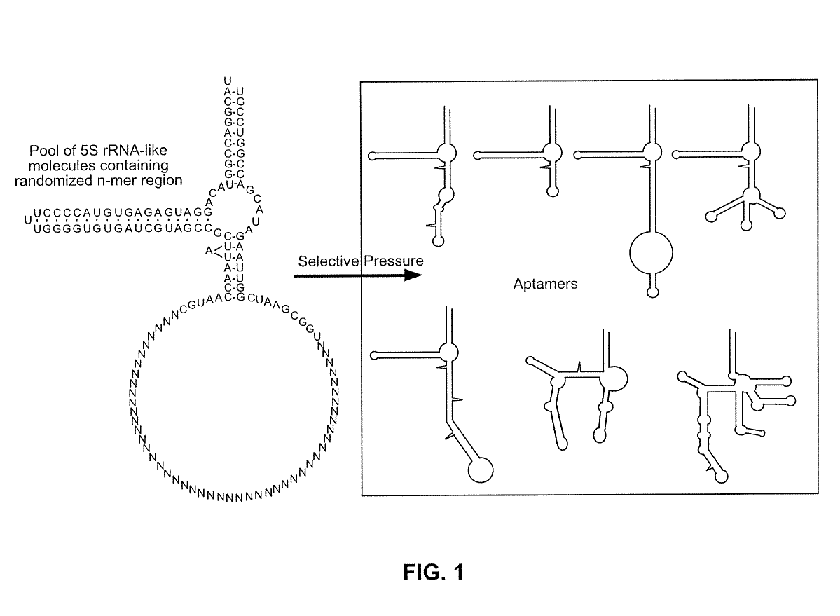

### DETAILED DESCRIPTION OF THE INVENTION

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of the present invention, the following terms are defined below.

An “aptamer” refers to a nucleic acid molecule that is capable of binding to a particular molecule of interest with high affinity and specificity (Tuerk and Gold, Science 249:505 (1990); Ellington and Szostak, Nature 346:818 (1990). The binding of a ligand to an aptamer, which is typically RNA, may also change the conformation of the aptamer and the nucleic acid within which the aptamer is located. The conformation change inhibits translation of an mRNA in which the aptamer is located, for example, or otherwise interferes with the normal activity of the nucleic acid. This type of interaction, with a small molecule metabolite, for example, coupled with subsequent changes in nucleic acid function has been referred to as a ‘riboswitch’. Aptamers may also be composed of DNA or may comprise non-natural nucleotides and nucleotide analogs. The method of selection may be by, but is not limited to, affinity chromatography and the method of amplification by reverse transcription (RT) or polymerase chain reaction (PCR).

Aptamers have specific binding regions which are capable of forming complexes with an intended target molecule in an environment wherein other substances in the same environment are not complexed to the nucleic acid. The specificity of the binding is defined in terms of the comparative dissociation constants (Kd) of the aptamer for its ligand as compared to the dissociation constant of the aptamer for other materials in the environment or unrelated molecules in general. Typically, the Kd for the aptamer with respect to its ligand will be at least about 10-fold less than the Kd for the aptamer with unrelated material or accompanying material in the environment. Even more preferably, the Kd will be at least about 50-fold less, more preferably at least about 100-fold less, and most preferably at least about 200-fold less.

An aptamer will typically be between about 10 and about 300 nucleotides in length. More commonly, an aptamer will be between about 30 and about 100 nucleotides in length.

The terms “nucleic acid molecule” and “polynucleotide” refer to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Cassol et al. (1992); Rossolini et al., Mol. Cell. Probes 8:91-98 (1994)). Also included are molecules having naturally occurring phosphodiester linkages as well as those having non-naturally occurring linkages, e.g., for stabilization purposes. The nucleic acid may be in any physical form, e.g., linear, circular, or supercoiled. The term nucleic acid is used interchangeably with oligonucleotide, gene, cDNA, and mRNA encoded by a gene.

A riboswitch is typically considered a part of an mRNA molecule that can directly bind a small target molecule, and whose binding of the target affects the gene's activity [Tucker BJ, Breaker RR (2005). “Riboswitches as versatile gene control elements”. Curr Opin Struct Biol 15 (3): 342-8]. Thus, an mRNA that contains a riboswitch is directly involved in regulating its own activity, depending on the presence or absence of its target molecule. By definition, then, a riboswitch has a region of aptamer-like affinity for a separate molecule. Thus, in the broader context of the instant invention, any aptamer included within a non-coding nucleic acid could be used for sequestration of molecules from bulk volumes. Downstream reporting of the event via “(ribo)switch” activity may be especially advantageous. A similar concept is coined by the phrase “aptazyme” in which an aptamer region is used as an allosteric control element and coupled to a region of catalytic RNA (a “ribozyme” as described below).

A ribozyme (from ribonucleic acid enzyme, also called RNA enzyme or catalytic RNA) is a RNA molecule that catalyzes a chemical reaction. Many natural ribozymes catalyze either the hydrolysis of one of their own phosphodiester bonds, or the hydrolysis of bonds in other RNAs, but they have also been found to catalyze the aminotransferase activity of the ribosome. More recently it has been shown that catalytic RNAs can be “evolved” by in vitro methods [1. Agresti J J, Kelly B T, Jäschke A, Griffiths A D: Selection of ribozymes that catalyse multiple-turnover Diels-Alder cycloadditions by using in vitro compartmentalization. Proc Natl Acad Sci USA 2005, 102:16170-16175; 2. Sooter L J, Riedel T, Davidson E A, Levy M, Cox J C, Ellington A D: Toward automated nucleic acid enzyme selection. Biological Chemistry 2001, 382(9):1327-1334.]. Winkler et al. have shown [Winkler W C, Nahvi A, Roth A, Collins J A, Breaker RR: Control of gene expression by a natural metabolite-responsive ribozyme. Nature 2004, 428:281-286.] that, similar to riboswitch activity discussed above, ribozymes and their reaction products can regulate gene expression. In the context of the instant invention, it may be particularly advantageous to place a catalytic RNA or ribozyme within a larger non-coding RNA such that the ribozyme is present at many copies within the cell for the purposes of chemical transformation of a molecule from a bulk volume. Furthermore, encoding both aptamers and ribozymes in the same non-coding RNA may be particularly advantageous.

The term “gene” is used broadly to refer to any segment of DNA associated with a biological function. Thus, genes include coding sequences and/or the regulatory sequences required for their expression. Genes can also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “bases” refers to both the deoxyribonucleic and ribonucleic acids. The following abbreviations are used, “A” refers to adenine as well as to it's deoxyribose derivative, “T” refers to thymine “U” refers to uridine, “G” refers to guanine as well as its deoxyribose derivative, “C” refers to cytosine as well as its deoxyribose derivative. A person having ordinary skill in this art would readily recognize that these bases may be modified or derivatized to optimize the methods of the present invention.

The invention is a novel methodology to sequester trace contaminants or target molecules from water and other process streams during biological treatment. More specifically, disclosed herein are methods of improving the removal or treatment of target molecules or contaminants in bulk volume by nucleic acid ligands generated to specifically bind to or alter target molecules or contaminants in bulk volumes. Genomic manipulation and selection of prokaryotic or eukaryotic cells will be used to place these nucleic acid ligands into naturally amplified non-coding nucleic acid sequences. By growing cells expressing random nucleic acid sequences, under increasing contaminant concentration, nucleic acid ligands will be selected in vivo that sequester these contaminants.

Bulk volumes refers to any volume of substance wherein the removal and/or treatment of substances therein occurs. Bulk volume includes waste fluids and/or streams, municipal waste and/or any other form of treatable waste.

Target molecules contemplated include but are not limited to metal ions, organic molecules, viral particles, biological molecules, such as antibodies, proteins, enzymes, pharmaceuticals and/or any other substance to be removed and/or treated from a bulk volume. In particular, wastes and contaminants are contemplated. Sequestration of target molecules refers to binding to or altering the target molecules.

Nucleic acid sequences, that can be utilized as discussed include but are not limited to aptamers, ribozymes, aptazymes, riboswitches, and/or any other nucleic acid sequence with particular binding and/or catalytic activity. For example, catalytic nucleic acids may be utilized to perform a treatment reaction, such as degradation, of a target molecule. Catalytic nucleic acids may also augment the catalytic action of other catalytic mechanisms, such as enzymatic catalytic mechanisms in cells.

Aptamers and/or other nucleic acids are generated to bind with relatively high affinity to a target molecule. Numerous methods of generating aptamers are known in the art. A common method of generating aptamers is known as the Systematic Evolution of Ligands by Exponential Enrichment or SELEX. In general, the process includes the synthesis of a large oligonucleotide library consisting of randomly generated sequences of fixed length flanked by constant 5′ and 3′ ends that serve as primers. For a randomly generated region of length n, the number of possible sequences in the library is 4′. The sequences in the library is then exposed to the target molecule and those that do not bind to the target are removed, such as by chromatography methods. The bound sequences are then eluted and amplified by polymerase chain reaction (PCR) to prepare for subsequent rounds of selection in which the stringency of the elution conditions are increased to identify the strongest-binding sequences.

The aptamer generation process can be performed in vitro or, in some exemplary embodiments, the process may be performed in vivo. An in vivo aptamer generation is performed utilizing a host organism. A host organism is useful in performing the amplification of nucleic acids as such processes are typically innate to all cells. Prokaryotic hosts such as bacteria or eukaryotic hosts are utilized. The criteria for selection of a host organism include ability to be easily cultured or grown as well as provide high production of nucleic acids. Examples of host organisms may include, but are not limited to, E. coli, Staphylococcus, Bacillus, Pseudomonas, Citrobacteia, Klebsilla, Rhodococcus, and/or any other appropriate organism, such as any number of eubacteria, archaea, fungi, plant, and/or mammalian cells. Combinations of organism hosts can also be utilized. The selection of an organism and/or combination of organisms is based on known and/or desirable interaction with a given application and/or target substance. For example, a number of organisms have been typically employed for bioremediation having natural catalytic properties for breakdown of specific contaminant types.

In some exemplary embodiments, a host organism is utilized to both evolve and/or produce a nucleic acid sequence with particular binding and/or catalytic activity. Cells containing a particular nucleic acid sequence are exposed to given concentrations of a target substance. Cells are then selected for a given reaction to the target substance, such as, for example, survival after exposure, and are further selected utilizing increasing concentrations of the substance. This method of selecting cells capable of generating and evolving a nucleic acid sequence is similar to in vitro SELEX. This method can be used for large groupings of different sequences for high-throughput.

Aptamers can integrate within the nucleic acid sequence of the host organism. Some nucleic acids, particularly RNA, are subject to rapid degradation in biological environments due to targeting by nuclease activity. However, it has been shown that some nucleic acid sequences are resistant to such degradation either by their inherent size or their similarity to structured RNAs within the cell. A degradation resistant nucleic acid sequence is utilized to protect an aptamer sequence from degradation in an organism and/or other biological environment. The aptamer sequence is inserted into an appropriate region of the degradation-resistant nucleic acid sequence subject to a high degree of molecular production, such as, for example, a ribosomal RNA (rRNA). In addition to rRNA, aptamers are inserted in other non-coding RNAs (i.e. RNAs which do not code for proteins) such as RNase P, tRNAs, small nuclear RNA (snRNA), small nucleolar RNAs (snoRNAs), efference RNA (eRNA), tmRNA, and/or any other appropriate nucleic acids recognized in molecular biology as “non-coding”. Many of these nucleic acids, while non-coding, have the capacity to accrue to significant levels within cells and are useful for high production of aptamers.

Nucleic acid sequences can be introduced in an organism by a variety of methods, such as, for example, transformation of a cell utilizing a nucleic acid construct, such as a plasmid. Nucleic acid sequences can also be incorporated into the host chromosome. The included nucleic acid sequence contains, aside from containing a nucleic acid sequence with particular binding and/or catalytic activity, other features, such as, for example, selection factors including antibiotic resistance genes, detection assay elements, controllable expression elements, and/or any other appropriate features.

Nucleic acid sequences with established and/or otherwise known ability to bind and/or otherwise interact with a target substance can also be utilized as a starting sequence. For example, known, well-characterized aptamers to ions, small inorganic or organic molecules, proteins, peptides, whole viral particles, and/or other appropriate targets are inserted into non-coding nucleic acid sequences for the purpose of sequestering such molecular targets. Such aptamers, identified by in vitro methods of selection have different binding affinity within the context of the surrounding non-coding nucleic acid, but they nevertheless retain significant affinity for their targets. Upstream (e.g. 5′-) and downstream (e.g. 3′-) polynucleotide spacers and/or appendages can be added to the known sequences to relax conformational constraints placed upon the sequences by tethering them within the context of the non-coding nucleic acid. Furthermore, multiple aptamers are concatenated to give increased avidity to the molecular target(s).

Bulk volumes can be treated with the genetically modified cells containing functional aptamer and/or catalytic nucleic acids within non-coding nucleic acid sequences. The genetically modified cells treat, remove and/or sequester target molecules in the bulk volume. The presence of a high concentration of binding and/or catalytic nucleic acids inside the cell creates an equilibrium shift in the bulk volume whereby a given substance is removed from the bulk volume and sequestered in the cell by binding to and/or catalytic action by the modified nucleic acids. The sequestration and/or catalytic action generally constantly removes the targeted molecule from the equilibrium, resulting in a constant influx of the target molecule into the cell. The genetically modified cells, harboring the sequestered target molecules, are then removed from the bulk volume. Appropriate methods of removal of the genetically modified cells include, but are not limited to, filtration, sedimentation, centrifugation (accelerated sedimentation), flocculation, adsorption, membrane filtration, biofilm formation, membrane bioreactor, and/or any other physical configuration otherwise known in the art as a bioreactor, used to separate the treated waste stream from the cells.

Such bioreactors also include in situ remediation techniques in which the genetically modified cells are released into a controlled volume of the environment. Sequestration and/or chemical transformation of contaminants then occurs before the controlled volume passes into another portion of the environment. This is particularly useful in examples where the cells are introduced into waste water and/or other waste streams which are in contact with the environment. The genetically modified cells can be immobilized for contact with a bulk volume while not being distributed into the volume. Immobilization techniques include but are not limited to, microbial mats, mineral amendments, polymer gel formulations, and/or any other appropriate immobilization technique or combination may be utilized. Genetically-modified cells can be tagged for identification such that they can be isolated from a particular environment. Additionally, the cells can be genetically modified to include features for their removal from an environment, such as, for example, a susceptibility factor to a particular substance, an affinity to a particular separation method, and/or any other appropriate removal method.

Further, the cells may also include features for increasing the sequestration rate of a substance in a bulk volume. For example, a molecular channel and/or transporter may be utilized to enhance transport of a substance across the cell membrane into the cell. Metal ion and/or other small ion transport molecules are known and can be incorporated by genetic modification of the cell. Additionally, the cells can be engineered to export the aptamer containing nucleic acids into bulk environment, for example, by including nucleic acid sequences encoding viral packaging and/or export signals. Reuptake of released nucleic acid bound to the target molecule can be engineered for example, by binding to cell surface receptors and/or any other appropriate method.

An aptamer expressed within the context of a larger non-coding nucleic acid can also be used to sequester a valuable substance. For example, copper obtained by “microbial leaching” accounts for more than 15 percent of the annual U.S. copper production. Genetically modified cells bearing aptamers capable of binding target molecules of value, as discussed above, can be utilized to sequester large amounts of valuable metals, hormones, biological drugs, and/or any other appropriate substance. The cells can be concentrated into a biosolid containing a large amount and/or concentration of a given substance.

Aptamers as discussed above can be utilized as affinity handles for purification. For example, a non-coding nucleic acid may contain a high-affinity aptamer handle as well as a sequence of therapeutic or diagnostic value. The desired high-value nucleic acid is readily purified by binding the aptamer portion. Aptamers to common chromatographic matrices such as agarose, Sephadex, Sepharose, as well as more specialized affinity resins with immobilized metals, antibodies, proteins, peptides, and/or any other appropriate affinity material can be utilized. Aptamers to such affinity ligands is developed by well established in vitro methods or by in vivo methods similar to those discussed above. Inserted aptamers fused to nucleic acids can be used for therapeutic and/or diagnostic functions, such as, for example, short-interfering RNAs (siRNAs), microRNAs (miRNAs), short-hairpin RNAs (shRNAs), antisense molecules, diagnostic probes or probe libraries, and/or aptamer inhibitors. Aptamer inhibitors can be developed to many important biological pathways such as G-coupled protein receptors, protein kinases, and/or any other appropriate pathways. The therapeutic nucleic acid with an aptamer affinity handle can be included in another nucleic acid sequence, such as the degradation resistant sequences and/or high production sequences discussed above. Aptamers utilized as affinity handles for molecules can be sequenced, probed by hybridization, and/or characterized by some other analytical technique, such as, for example, sequencing or mass spectrometry for organism identification.

Inserted nucleic acid sequences are also useful for highly specific intracellular labeling and/or cellular signal tracking. For example, an aptamer including a fluorescent- and/or radio-label can be concatenated and/or fused to an aptamer targeting a particular cellular component, such as an important protein, enzyme, organelle, and/or any other appropriate component. This aptamer fusion can be expressed at high levels within a non-coding nucleic acid, as described above. Cells expressing such aptamers may thus have a built-in ability to monitor specific cellular processes.

In another embodiment, an initial randomized library may be inserted in a non-coding nucleic acid sequence and selected in vitro for certain interaction and/or catalytic activity while contained within a reverse phase emulsion, a method referred to as in vitro compartmentalization. In such a process, on average one template encoding an aptamer would be accommodated within the reverse phase micelle. For those micelles in which the desired catalytic activity was achieved, an affinity handle, such as biotin, may be attached to the encoding gene. Catalytic ribozymes, aptazymes, and/or other catalytic nucleic acids may then be encoded within the context of a larger gene encoding a non-coding nucleic acid, such as rRNA. Affinity handles may then be added to those genes, which may be individually compartmentalized in micelles, which encode nucleic acids with the desired catalytic activity.

Hence, disclosed herein is an expression vector comprising a chimeric gene encoding selective nucleic acid ligands within a non-coding nucleic acid, operatively linked to a functional promoter, where the vector when transfected into a host transcribes the chimeric gene, and where the gene product is capable of binding to or altering target molecules. In an embodiment, the vector further comprises a selection marker. Specifically, the selection marker is an antibiotic resistance marker. Moreover, the promoter is a T7 RNA polymerase or a ribosomal RNA promoter. In general, the nucleic acid ligands are an aptamer, ribozyme, aptazyme or a riboswitch. Additionally, the non-coding nucleic acid is selected from the group consisting of rRNA, tRNA, RNAase P, small nuclear RNA (snRNA), small nucleolar RNA (snoRNA), efference RNA (eRNA) and tmRNA. In general, the target molecules are waste water contaminants. Specifically, the waste water contaminants are inorganic molecules, organic molecules, toxins, proteins, peptides, or viral particles. In one embodiment, the target molecules are hormones, antibodies, proteins, enzymes, pharmaceuticals or valuable metals. Further disclosed is an isolated cell comprising the expression vector described supra. In general, the cell is a prokaryotic cell or a eukaryotic cell.

In another embodiment of the present invention there is an isolated cell comprising at least one nucleic acid ligand sequence, incorporated into a genomic non-coding nucleic acid, where the nucleic acid ligand sequence binds to or catalytically alters the target molecule. In general, the nucleic acid ligand sequence is incorporated into the genomic non-coding nucleic acid by homologous recombination. The non-coding nucleic acid is selected from the group consisting of rRNA, tRNA, RNAase P, small nuclear RNA (snRNA), small nucleolar RNA (snoRNA), efference RNA (eRNA) and tmRNA. In general, the cell is a prokaryotic cell or a eukaryotic cell. Moreover, the nucleic acid sequence is an aptamer, ribozyme, aptazyme or a riboswitch. In general, the target molecule is a waste water contaminant or substance otherwise desired to be sequestered from the treatment stream. Specifically, the waste water contaminants are inorganic molecules, organic molecules, toxins, proteins, peptides, or viral particles. In a related embodiment the target molecule is a hormone, antibody, protein, enzyme, or a valuable metal.

In yet another embodiment, there is provided a method for sequestering within a cell a plurality of target molecules, present in a bulk volume comprising, generating a library of nucleic acid ligand sequences capable of binding to the target molecules; incorporating the nucleic acid ligand sequences into at least one non-coding nucleic acid within a cell; culturing the cell to achieve a cell population; Contacting the cell population with the bulk volume; and separating the cell population from the bulk volume. The method further comprises recovering the target molecule from the cell population. In general, the target molecules are inorganic molecules, organic molecules, toxins, proteins, peptides, and viral particles. In a related embodiment, the target molecules are hormones, antibodies, proteins, enzymes, pharmaceuticals or valuable metals. In general, the separation is accomplished by a method selected from the group consisting of filtration, sedimentation, flocculation, adsorption, membrane filtration, biofilm formation and membrane bioreactor interaction. The non-coding nucleic acid is selected from the group consisting of rRNA, tRNA, RNAase P, small nuclear RNA (snRNA), small nucleolar RNA (snoRNA), efference RNA (eRNA) and tmRNA. Specifically, the incorporation of nucleic acid ligand sequence is into the genomic non-coding nucleic acid. The nucleic acid sequence is an aptamer, ribozyme, aptazyme or a riboswitch. Moreover, the cell is a prokaryotic or a eukaryotic cell.

In yet another embodiment of the present invention, there is provided a method for bioremediation of contaminants present in a bulk volume comprising, generating a library of nucleic acid ligand sequences capable of binding to or altering the contaminants; incorporating the nucleic acid ligand sequences in at least one non-coding nucleic acid in a cell; culturing the cell to achieve a cell population; contacting the cell population with the bulk volume; and separating the cell population from the bulk volume. In general, the contaminants are inorganic molecules, organic molecules, toxins, proteins, peptides, and viral particles. The bulk volume is bodily waste fluids, municipal waste water or effluent from an industrial process.

Accordingly, various embodiments of the present invention disclose an expression vector comprising:a chimeric gene encoding selective nucleic acid ligands within a non-coding nucleic acid, operatively linked to a functional promoter, wherein said expression vector when transfected into a host transcribes said chimeric gene, and wherein said gene product is capable of binding to or altering at least one target molecule.

Further embodiments disclose an isolated cell comprising: at least one nucleic acid ligand sequence incorporated into a genomic DNA encoding a non-coding nucleic acid, wherein said nucleic acid ligand sequence binds to or catalytically alters a target molecule.

Yet further embodiments disclose a method for sequestering within a cell a plurality of target molecules, present in a bulk volume comprising: generating a library of nucleic acid ligand sequences capable of binding to said target molecules; incorporating said nucleic acid ligand sequences into at least one non-coding nucleic acid within a cell; culturing said cell to achieve a cell population; contacting said cell population with said bulk volume; and separating said cell population from said bulk volume.

Various other embodiments disclose methods for bioremediation of at least one contaminant present in a bulk volume comprising generating a library of nucleic acid ligand sequences capable of binding to or altering said at least one contaminant; incorporating said nucleic acid ligand sequences in at least one non-coding nucleic acid in a cell; culturing said cell to achieve a cell population; contacting said cell population with said bulk volume; and, separating said cell population from said bulk volume.

The following examples are given for the purpose of illustrating various embodiments of the invention and are not meant to limit the present invention in any fashion.

### Example 1

### Chromosomal Modification for Encoding Randomized Sequences within Modified rRNA

A plasmid-based system for expressing random libraries of RNA sequences within the context of a larger 5S rRNA sequence will be used. While this system has some advantages in terms of being selectively inducible by IPTG and can be used to readily identify new aptamers, the desired strains should be chromosomal variants. This is mainly because for the water or solids waste applications, it would be undesirable to maintain a plasmid system by the continual addition of an antibiotic. Described below are steps to create a very similar system residing on the chromosome of E. coli.

In order to introduce the necessary genetic modifications the protocol as described in Ammons et al. will be followed. See Ammons D, Rampersad J, Fox GE: A Genomically Modified Marker Strain of Escherichia coli. Current Microbiology 1998, 37:341-346. The aRNAs containing the random RNA libraries within the 5S rRNA will be subcloned into the plasmid containing the recombination cassette, pKO3-SARP. The host strain for integration of the artificial RNA will be the recombination proficient E. coli strain EMG2 (F′, lambda+). Excision of the kanamycin cassette will be performed with the aid of the Saccharomyces cerevisiae gene coding for Flp recombinase (FLP), contained in the pCP20 plasmid, and the Flp DNA target sequence (FRT), present in pKO3-SARP.

### Example 2

### Chromosomal Integration of the Randomized aRNA Library

The protocol to be used for gene replacement will be as described in Ammons et al. See Ammons D, Rampersad J, Fox G E: A Genomically Modified Marker Strain of Escherichia coli. Current Microbiology 1998, 37:341-346, which has been derived from Link et al. See Link et al Methods for Generating Precise Deletions and Insertions in the Genome of Wild-Type Escherichia coli: Application to Open Reading Frame Characterization. Journal of Bacteriology 1997, 179(20):6228-6237. Briefly, EMG2 cells are transformed with the pKO3-SARP-derived plasmids containing the aRNA library, obtained by subcloning. A single transformation colony is then plated on a yeast tryptone (YT) agar plate containing chloramphenicol (80 ug/ml) and incubated at 42° C. The pKO3-SARP plasmid confers chloramphenicol resistance but is temperature sensitive and thus, cannot replicate at 42° C. Only the cells in which the plasmid has integrated into the chromosome will be able to grow in presence of the antibiotic. A single colony is then plated onto a YT plate containing kanamycin (50 ug/ml) and sucrose (5% w/v), and incubated at 30° C., at which a plasmid can replicate. The host plasmid also contains the Bacillus subtilis sacB gene, whose gene product kills E. coli cells grown in the presence of sucrose. At 30° C., cells that contain a chromosomal copy of the plasmid cannot grow efficiently. Thus, the colonies that do grow result from a second recombination event in which the plasmid containing the exchanged host 5S rRNA gene has been excised from the chromosome. The resulting cell line is further transformed with plasmid pCP20. This plasmid is also temperature sensitive for replication as for FLP expression and at 42° C. both expresses Flp recombinase and ceases to replicate, resulting in the excision of the kanamycin gene from between the FRT flanking sites in the chromosome and loss of the pCP20 plasmid.

### Example 3

### PCR, Cloning, and Sequencing for Verification of Random Library Strains

To verify that our randomized aRNA library has been inserted into the E. coli chromosome several primers that have been described in Ammons et al. will be used. Primers A (CCCGAGACTCAGTGAAATTG) (SEQ ID NO:1), B (CCCAAGAATTCATATCGACGGC) (SEQ ID NO:1), C(CCCAAGCTTCGCTACTGCCGCCAGGCA) (SEQ ID NO:3), and D (TCCCCCGGGAGTAGGGAACTGCCAGGCAT) (SEQ ID NO:4) are nonspecific and hybridize to orthologous regions present in all seven rRNA operons. Primer E (GGCTCTCTTTCAGACTTGGG) (SEQ ID NO:5) is specific for rRNA operon H. PCR amplification using primers A and E will allow for the discrimination between an aRNA gene insertion and a wild type 5S rRNA gene and this will be evident by standard electrophoretic analysis. The amplified sequence would then be cloned using the TOPO TA system (Invitrogen) to be subsequently sequenced with the expectation that the randomized region will generate many indeterminate base-calls or “Ns” by sequencing.

### Example 4

### Quantification of Library Expression Levels

To quantify the relative amount of the aRNA library being expressed, total RNA from cells in log-phase growth using a standardized Trizol™ reagent (Invitrogen) or similar “home brew” version will be isolated. See Chomczynski P, Sacchi N: Single Step Method of RNA Isolation by Acid Guanidinium Thiocyanate-Phenol-Chloroform Extraction. Anal Biochem 1987, 162:156. Following a 30 nt deletion and 50 nt insertion of randomized sequence, the final “5S-like” RNA pool for in vivo selection will be 140 nt long. This is 20 nt longer than 5S rRNA, the nearest major RNA species to be isolated by the Trizol method. It will therefore be straightforward to separate this RNA library of interest from the native RNAs by standard electrophoretic methods and quantify the relative expression of the library. The relative expression will be characterized at various times in the growth cycle of the cells and for various growth conditions.

### Example 5

### In Vivo Selection of Nickel Aptamers

While nickel is referred to as a heavy metal, it is relatively safe to work with. Novel genomically modified strains expressing randomized 50-mer ribosomal-inserts, will be cultured in the presence of increasing NiCl₂ concentrations. Strains capable of growth within at least 10 mM Ni²⁺ are expected to evolve. This represents a heavily contaminated water stream at over 100 parts per million (ppm) levels or 6.022×10¹⁸ nickel atoms/ml. At a minimum a culture containing ˜1×10⁹ cells/ml each containing 50,000 ribosomes, is expected to impart this level of tolerance . If 1/7 of these ribosomes contain an aptamer the culture will contain ˜7×10¹² aptamers/ml. Thus, at 10 mM, it is likely that the nickel ions are in excess to the evolved aptamer ligands, yet it is apparent from these numerical estimates that ribosomal aptamers are being produced in large numbers and impart increased fitness to the organism in the presence of contaminant.

### Example 6

### In vivo Selection of Malachite Green, Luciferin, and β-estradiol Aptamers

Malachite green is a widely used triphenylmethane dye with known ability to cross into the cell cytoplasm. Further, Babendure, et al. have selected aptamers in vitro against malachite green and related molecules which, when bound to the dye, cause a tremendous fluorescence enhancement (greater than 2300-fold) that is readily detectable by spectroscopy and/or fluorescence microscopy. Whether or not this fluorescence enhancement occurs when bound to our in vivo selected aptamers, malachite green should be easily detectable both inside and outside of cells. This feature will be especially useful in demonstrating that the novel strains of the present invention improve sequestration of contaminants inside of cells. Malachite green is also interesting as an organic molecule of similar molecular weight as that of many problematic water contaminants, and variants of the dye with halogen and other substitutions are available. Finally, the compound itself is a known carcinogen and is used as an antimicrobial agent in aquaculture in some parts of the world. Development of ribosomal aptamers to malachite green may therefore have intrinsic value in its own right.

Luciferin is the substrate of the enzyme luciferase. Luciferin is also similar in molecular weight to many toxins, pharmaceuticals, pesticides, and hormones. Ribosomal-aptamers to luciferin, developed will aid in assaying for its sequestration within E. coli by the use of luciferase, the well known light-generating firefly enzyme.

β-estradiol is the major estrogen secreted by the pre-menopausal ovary. Exposure to estradiol increases breast cancer incidence and proliferation. Increasing evidence is mounting that estrogens and mimics are accumulating in the environment with detrimental effects on a variety of plants and animals, including humans.

First, the toxic limits of malachite green, luciferin, and β-estradiol for unmodified strains of E. coli will be determined. The random library strain developed supra will be cultured under increasing concentrations of the three model contaminants. Any culturing scheme in which the successive generations of cells are exposed to increasing contaminant pressure (concentration) should be suitable for the purposes of directing evolution of the random library to specific aptamer sequences. Especially in the case of malachite green, some significant portion of total contaminant is expected to be sequestered in the inner membrane (peptidoglycan) region of E. coli. The dye has been routinely used to stain bacterial endospores within cells of species such as Bacillus anthracis. Hence, some fraction of the malachite green will be available in the cytosol and some bulk concentration will induce selective pressure to develop aptamers to the compound. As with malachite green, it is expected that at some high concentration, growth will be inhibited thereby ensuring that the molecule is available in the cytosol and selective pressure is applied to the random ribosomal library.

### Example 9

### To Demonstrate Improved Sequestration of Contaminants from Water Using Our Novel Bacterial Strains Containing Ribosomal Aptamers

Having evolved new ribosomal-aptamers to model water contaminants in vivo and characterized their binding affinity in vitro, the ultimate utility of our approach for facilitating contaminant clearance from water streams will be demonstrated. The assumption is that in most applications, sequestration of trace molecular contaminants (whether they are subsequently degraded or not) within cells will facilitate their removal either by mechanical filtration of cells or settling. In contrast to the in vitro experiments involving only purified aRNA, these experiments will use whole E. coli cells containing ribosomal-aptamers. To demonstrate the enabling nature of ribosomal-aptamers, several qualitative and quantitative partitioning experiments using model contaminant targets will be performed.

### Mechanical Filtration and Nickel Quantitation for Nickel Partitioning Measurement

To demonstrate sequestration of the heavy metal ion Ni²⁺, the nickel aptamer strain developed above will be cultured in the presence of increasing concentrations of NiCl₂. Immediately following culture to log-phase growth (OD₆₀₀ ≈1.0) the cell suspensions will be mechanically filtered using commercially available 0.2 μm syringe filters. The nickel will be then quantified in the filtrate as described by spectrophotometry. If necessary for reliable spectrophotometric quantitation, nickel calibration curve in the optically-clear culture medium M9 and culture strains in that medium (with any necessary supplementation) will be developed. As a control, the same experiments using standard strains of E. coli with no ribosomal-aptamers will be performed. Any nickel retention by the control strain will be “subtracted off” of the results for the nickel aptamer strain prior to calculation of partitioning coefficients. Partitioning values will be calculated in triplicate for approximately 7 nickel concentrations.

### Centrifugation and Supernatant Analysis for Malachite Green, Luciferin, and ¹⁴C-Estrogen Partitioning Measurement

Similar to the above nickel experiment, E. coli containing aptamers and mechanical filtration to mechanically sequester model organic contaminants will be used. One concern, however, is that some amount of these organics might bind to the syringe membrane filters (typically PVDF, however we may investigate alternatives). To avoid this complication, the cell suspension will be partitioned by centrifugation. The supernatants will then be analyzed for malachite green, luciferin, and β-estradiol as described supra. As in the nickel partitioning determination, baseline sequestration of these organics using a control strain (which is likely to be more significant due to hold-up in the inner membrane of E. coli) will be subtracted before determination of partitioning coefficients.

### Fluorescence Microscopy of Increased Malachite Green Sequestration

Malachite green aptamer strains and control strains of E. coli will be bathed in several concentrations of malachite green, cells will be affixed to slides using standard methods, and examined using either fluorescence microscopy or fluorescence imaging. To decrease, background fluorescence (if necessary) cells will be cultured on membranes (Neogen, Inc) placed on agar containing malachite green. The membrane will be transferred to agar containing activated carbon. Excess malachite green will therefore be “destained” with the expectation that the aptamer-containing cells will fluoresce with much more intensity than the control strain.

The invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes to the claims that come within the meaning and range of equivalency of the claims are to be embraced within their scope. Further, all published documents, patents, and applications mentioned herein are hereby incorporated by reference, as if presented in their entirety.

What is claimed is:

1. An expression vector comprising:
a chimeric gene encoding selective nucleic acid ligands within a non-coding nucleic acid, operatively linked to a functional promoter, wherein said expression vector when transfected into a host transcribes said chimeric gene, and wherein said gene product is capable of binding to or altering at least one target molecule.

2. The expression vector of claim 1, wherein said vector further comprises at least one of a selection marker or a marker for selective induction.

3. The expression vector of claim 2, wherein said vector further comprises at least one selection marker and wherein said selection marker is an antibiotic resistance marker.

4. The expression vector of claim 1, wherein said promoter is a T7 RNA polymerase or a ribosomal RNA promoter.

5. The expression vector of claim 1, wherein said selective nucleic acid ligands are at least one of an aptamer, a ribozyme, an aptazyme or a riboswitch.

6. The expression vector of claim 1, wherein said non-coding nucleic acid is selected from the group consisting of rRNA, tRNA, RNAase P, small nuclear RNA (snRNA), small nucleolar RNA (snoRNA), efference RNA (eRNA) and tmRNA.

7. The expression vector of claim 1, wherein said at least one target molecule is a waste fluid contaminant.

8. The expression vector of claim 7, wherein said waste fluid contaminant is at least one of an inorganic molecule, an organic molecule, a toxin, a peptide, or a viral particle.

9. The expression vector of claim 1, wherein said at least one target molecule is at least one of hormones, antibodies, proteins, enzymes, pharmaceuticals or metals.

10. An isolated cell comprising said expression vector of claim 1.

11. The cell of claim 10, wherein said cell is a prokaryotic cell or a eukaryotic cell.

12. An isolated cell comprising:
at least one nucleic acid ligand sequence incorporated into a genomic DNA encoding a non-coding nucleic acid, wherein said nucleic acid ligand sequence binds to or catalytically alters a target molecule.

13. The cell of claim 12, wherein said nucleic acid ligand sequence is incorporated into said non-coding nucleic acid by homologous recombination.

14. The cell of claim 12, wherein said non-coding nucleic acid is selected from the group consisting of rRNA, tRNA, RNAase P, small nuclear RNA (snRNA), small nucleolar RNA (snoRNA), efference RNA (eRNA) and tmRNA.

15. The cell of claim 12, wherein said cell is a prokaryotic cell or a eukaryotic cell.

16. The cell of claim 12, wherein said nucleic acid sequence is an aptamer, a ribozyme, an aptazyme or a riboswitch.

17. The cell of claim 12, wherein said target molecule is a waste fluid contaminant.

18. The cell of claim 17, wherein said waste fluid contaminant is an inorganic molecule, an organic molecule, a toxin, a peptide, or a viral particle.

19. The cell of claim 12, wherein said target molecule is a hormone, an antibody, a protein, an enzyme, or a metal.

20. A method for sequestering within a cell a plurality of target molecules, present in a bulk volume comprising:
generating a library of nucleic acid ligand sequences capable of binding to said target molecules;
incorporating said nucleic acid ligand sequences into at least one non-coding nucleic acid within a cell;
culturing said cell to achieve a cell population;
contacting said cell population with said bulk volume; and
separating said cell population from said bulk volume.

21. The method of claim 20, further comprising recovering said target molecule from said cell population.

22. The method of claim 20, wherein said target molecules are at least one of an inorganic molecule, an organic molecule, a toxin, a peptide, a viral particle, a hormone, an antibody, a protein, an enzyme, a pharmaceutical or a metal.

23. The method of claim 20, wherein said separation is accomplished by a method selected from the group consisting of filtration, sedimentation, flocculation, adsorption, membrane filtration, biofilm formation and membrane bioreactor interaction.

24. The method of claim 20, wherein said non-coding nucleic acid is selected from the group consisting of rRNA, tRNA, RNAase P, small nuclear RNA (snRNA), small nucleolar RNA (snoRNA), efference RNA (eRNA) and tmRNA.

25. The method of claim 20, wherein said incorporation of said nucleic acid ligand sequence is into said genomic DNA encoding said non-coding nucleic acid.

26. The method of claim 20, wherein said nucleic acid sequence is an aptamer, a ribozyme, an aptazyme or a riboswitch.

27. The method of claim 20, wherein said cell is a prokaryotic or a eukaryotic cell.

28. A method for bioremediation of at least one contaminant present in a bulk volume comprising:
generating a library of nucleic acid ligand sequences capable of binding to or altering said at least one contaminant;
incorporating said nucleic acid ligand sequences in at least one non-coding nucleic acid in a cell;
culturing said cell to achieve a cell population;
contacting said cell population with said bulk volume; and,
separating said cell population from said bulk volume.

29. The method of claim 29, wherein said contaminants are at least one of an inorganic molecule, an organic molecule, a toxin, a protein, a peptide, and a viral particle.

30. The method of claim 29, wherein said bulk volume is at least one of bodily waste fluids, municipal waste water or effluent from an industrial process.


SEQUENCE LISTING (5 sequences)

SEQ ID NO: 1 - DNA, 20 bp, Escherichia Coli
cccgagactcagtgaaattg

SEQ ID NO: 2 - DNA, 22 bp, Escherichia coli
cccaagaattcatatcgacggc

SEQ ID NO: 3 - DNA, 27 bp, Escherichia coli
cccaagcttcgctactgccgccaggca

SEQ ID NO: 4 - DNA, 29 bp, Escherichia coli
tcccccgggagtagggaactgccaggcat

SEQ ID NO: 5 - DNA, 20 bp, Escherichia coli
ggctctctttcagacttggg